(19) United States
(12) Patent Application Publication
(10) Pub. No.: US 2012/0283621 A1
(43) Pub. Date: Nov. 8, 2012

(54) STERILIZING APPLICATION OF CROSS-LINKING AGENT

(75) Inventor: David Muller, Boston, MA (US)

(73) Assignee: Avedro, Inc., Waltham, MA (US)

(21) Appl. No.: 13/468,760

(22) Filed: May 10, 2012

Related U.S. Application Data
(63) Continuation-in-part of application No. 13/051,699, filed on Mar. 18, 2011.
(60) Provisional application No. 61/315,840, filed on Mar. 19, 2010.
(60) Provisional application No. 61/319,111, filed on Mar. 30, 2010.
(60) Provisional application No. 61/326,527, filed on Apr. 21, 2010.
(60) Provisional application No. 61/328,138, filed on Apr. 26, 2010.
(60) Provisional application No. 61/377,024, filed on Aug. 25, 2010.
(60) Provisional application No. 61/388,963, filed on Oct. 1, 2010.
(60) Provisional application No. 61/409,103, filed on Nov. 1, 2010.
(60) Provisional application No. 61/423,375, filed on Dec. 15, 2010.
(60) Provisional application No. 61/484,572, filed on May 10, 2011.

(51) Int. Cl. A61M 37/00

(52) U.S. Cl. 604/20

(57) ABSTRACT

A method for treating an eye includes applying, to an outer protective layer of an area of an eye, one or more initial doses of a cross-linking agent that acts as a photosensitizer. The method also includes delivering, from a light source, one or more initial doses of ultraviolet light to the area of the eye. The cross-linking agent increases absorption of the ultraviolet light by the area of the eye, and the absorption of the ultraviolet light sterilizes the area of the eye before the outer protective layer is penetrated. Additionally, the method includes penetrating the outer protective layer of the eye to provide access to an area below the outer protective layer. Moreover, the method includes applying a treatment to the eye via the access provided by cutting the outer protective layer. In some embodiments, the cross-linking agent is Riboflavin, and the treatment is LASIK surgery.

### CROSS REFERENCE TO RELATED APPLICATIONS

This application is a continuation-in-part of U.S. patent application Ser. No. 13/051,699, filed Mar. 18, 2011, which claims priority to U.S. Provisional Application No. 61/315,840, filed Mar. 19, 2010; U.S. Provisional Application No. 61/319,111, filed Mar. 30, 2010; U.S. Provisional Application No. 61/326,527, filed Apr. 21, 2010; U.S. Provisional Application No. 61/328,138, filed Apr. 26, 2010; U.S. Provisional Application No. 61/377,024, filed Aug. 25, 2010; U.S. Provisional Application No. 61/388,963, filed Oct. 1, 2010; U.S. Provisional Application No. 61/409,103, filed Nov. 1, 2010; and U.S. Provisional Application No. 61/423,375, filed Dec. 15, 2010, the contents of these applications being incorporated entirely herein by reference. This application also claims the benefit of, and priority to, U.S. Provisional Patent Application No. 61/484,572, filed May 10, 2011, the contents of which are incorporated entirely herein by reference.

### BACKGROUND OF THE INVENTION

1. Field of the Invention

The invention pertains to the field of medical treatment and, more particularly, to systems and methods for sterilizing a field, particularly on an eye, for a medical treatment, such as LASIK surgery.

2. Description of Related Art

Laser-assisted in-situ keratomileusis (LASIK) is one of a number of surgical procedures for treating various eye disorders. During LASIK surgery, an instrument called a microkeratome is used to cut through the corneal epithelium to form a thin flap. The flap is then peeled back and the underlying cornea tissue is reshaped using an excimer laser so that light traveling through the cornea is properly focused onto the retina.

The epithelium is the cornea's outermost region and one of its functions includes protecting the underlying tissue from exposure to foreign material, such as dust, water, and bacteria. As a result, the protective function of the epithelium is compromised when the microkeratome cuts through the epithelium during LASIK surgery. In general, any incision, injection, or other penetration of the outer protective layer, e.g., epithelium, during an eye treatment, e.g., LASIK surgery, increases the risk of infection.

### BRIEF SUMMARY

Embodiments according to aspects of the present invention provide systems and methods for reducing the risk of infection when eye treatments, e.g., LASIK surgery, require incision, injection, or other penetration of the outer protective layer of the eye.

According to an example embodiment, a method for treating an eye includes applying, to an outer protective layer of an area of an eye, one or more initial doses of a cross-linking agent that acts as a photosensitizer. The method also includes delivering, from a light source, one or more initial doses of ultraviolet light to the area of the eye. The cross-linking agent increases absorption of the ultraviolet light by the area of the eye, and the absorption of the ultraviolet light sterilizes the area of the eye before the outer protective layer is penetrated. Additionally, the method includes penetrating the outer protective layer of the eye to provide access to an area below the outer protective layer. Moreover, the method includes applying a treatment to the eye via the access provided by cutting the outer protective layer. In some embodiments, the cross-linking agent is Riboflavin.

According to further embodiments, the method includes delivering, from the light source, one or more additional doses of the ultraviolet light after penetrating the outer protective layer of the eye. The one or more additional doses of the ultraviolet light further sterilize the area of the eye. According to yet further embodiments, the method includes applying, one or more additional doses of the cross-linking agent after penetrating the outer protective layer of the eye. The one or more additional doses of the cross-linking agent increases absorption of the ultraviolet light by the area of the eye, and the absorption of the ultraviolet light further sterilizes the area of the eye

In particular embodiments, the treatment is LASIK surgery. Thus, in some embodiments, penetrating the outer protective layer of the eye includes cutting a thin flap from the outer protective layer of a cornea, and applying the treatment to the eye includes reshaping the cornea with an excimer laser while the thin flap is peeled back.

These and other aspects of the present disclosure will become more apparent from the following detailed description of embodiments of the present disclosure when viewed in conjunction with the accompanying drawings.

### BRIEF DESCRIPTION OF THE DRAWINGS

FIG. 1 provides a block diagram of an example delivery system for delivering a cross-linking agent and an activator to a cornea of an eye in order to initiate molecular cross-linking of corneal collagen within the cornea, according to aspects of the present disclosure.

FIG. 2A provides a flowchart showing an example embodiment for activating cross-linking within cornea tissue using a cross-linking agent and an initiating element, according to aspects of the present disclosure.

FIG. 2B provides a flowchart similar to FIG. 2A where Riboflavin may be applied topically as the cross-linking agent and UV light may be applied as the initiating element, according to aspects of the present disclosure.

FIG. 3A illustrates an example approach for stabilizing changes in corneal structure after LASIK treatment, according to aspects of the present disclosure.

FIG. 3B illustrates another example approach for stabilizing changes in corneal structure after LASIK treatment, according to aspects of the present disclosure.

FIG. 4 illustrates an example system for stabilizing changes in corneal structure after eye treatment, according to aspects of the present disclosure.

FIG. 5 illustrates an example approach for sterilizing a field on an eye prior to eye treatment according to aspects of the present invention.

FIG. 6 illustrates another example approach for sterilizing a field on an eye prior to eye treatment, according to aspects of the present disclosure.

FIG. 7 illustrates a further example approach for sterilizing a field on an eye prior to, and during, eye treatment, according to aspects of the present disclosure.

FIG. 8 illustrates a yet another example approach for sterilizing a field on an eye prior to, and during, eye treatment, according to aspects of the present disclosure.

FIG. 9 illustrates a device for sterilizing a field on an eye prior to, and during, eye treatment, according to aspects of the present disclosure.

### DETAILED DESCRIPTION

According to aspects of the present disclosure, a cross-linking agent is applied to the regions of the cornea treated by LASIK surgery, or other similar eye treatment. According to some embodiments, the cross-linking agent is applied to initiate molecular cross-linking of corneal collagen to stabilize corneal tissue and improve its biomechanical strength when LASIK surgery is employed to make corrections to corneal structure and shape. According to other embodiments, the cross-linking agent is applied to sterilize the field for LASIK surgery and reduce the risk of infection associated with penetration of the outer protective layer, e.g., epithelium, of the eye.

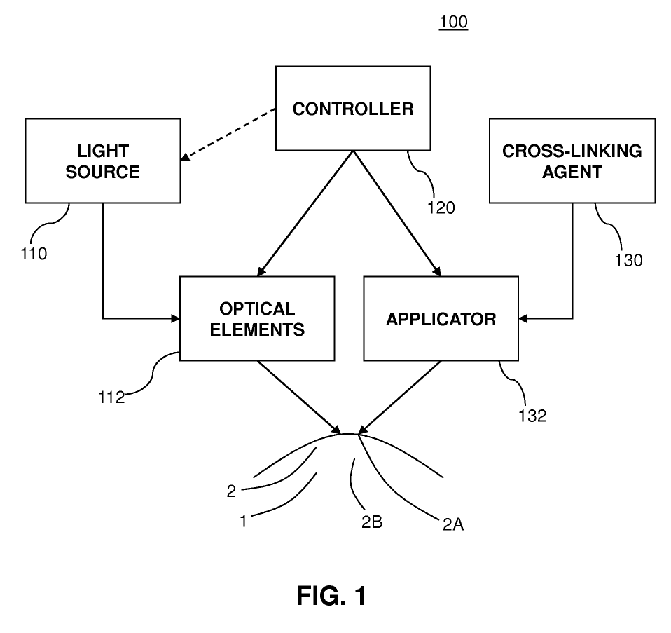

FIG. 1 provides a block diagram of an example delivery system 100 for delivering a cross-linking agent 130 and an initiating element, e.g., light, to a cornea 2 of an eye 1 in order to initiate molecular cross-linking of corneal collagen within the cornea 2. Cross-linking can stabilize corneal tissue and improve its biomechanical strength. The delivery system 100 includes an applicator 132 for applying the cross-linking agent 130 to the cornea 2. The delivery system 100 includes a light source 110 and optical elements 112 for directing light to the cornea 2. The delivery system 100 also includes a controller 120 that is coupled to the applicator 132 and the optical elements 112. The applicator 132 may be an apparatus adapted to apply the cross-linking agent 130 according to particular patterns on the cornea 2 where cross-linking activity may be more advantageous.

The optical elements 112 may include, for example, one or more mirrors or lenses for directing and focusing the light emitted by the light source 110 to a particular pattern on the cornea 2 suitable for activating the cross-linking agent 130. The light source 110 may be an ultraviolet light source, and the light directed to the cornea 2 through the optical elements 112 activates the cross-linking agent 130. The light source 110 may also alternatively or additionally emit photons with greater or lesser energy levels than ultraviolet light photons. The delivery system 100 also includes a controller 120 for controlling the operation of the optical elements 112 or the applicator 132, or both. By controlling aspects of the operation of the optical elements 112 and the applicator 132, the controller 120 can control the regions of the cornea 2 that receive the cross-linking agent 130 and that are exposed to the light source 110. By controlling the regions of the cornea 2 that receive the cross-linking agent 130 and the light source 110, the controller 120 can control the particular regions of the cornea 2 that are strengthened and stabilized through cross-linking of the corneal collagen fibrils. In an implementation, the cross-linking agent 130 can be applied generally to the eye 1, without regard to a particular region of the cornea 2 requiring strengthening, but the light source 110 can be directed to a particular region of the cornea 2 requiring strengthening, and thereby control the region of the cornea 2 wherein cross-linking is initiated by controlling the regions of the cornea 2 that are exposed to the light source 110.

The optical elements 112 can be used to focus the light emitted by the light source 110 to a particular focal plane within the cornea 2, such as a focal plane that includes the mid-depth region 2B. In addition, according to particular embodiments, the optical elements 112 may include one or more beam splitters for dividing a beam of light emitted by the light source 110, and may include one or more heat sinks for absorbing light emitted by the light source 110. The optical elements 112 may further include filters for partially blocking wavelengths of light emitted by the light source 110 and for advantageously selecting particular wavelengths of light to be directed to the cornea 2 for activating the cross-linking agent 130. The controller 120 can also be adapted to control the light source 110 by, for example, toggling a power switch of the light source 110.

In an implementation, the controller 120 may include hardware and/or software elements, and may be a computer. The controller 120 may include a processor, a memory storage, a microcontroller, digital logic elements, software running on a computer processor, or any combination thereof. In an alternative implementation of the delivery system 100 shown in FIG. 1, the controller 120 may be replaced by two or more separate controllers or processors. For example, one controller may be used to control the operation of the applicator 132, and thereby control the precise rate and location of the application of the cross-linking agent 130 to the cornea 2. Another controller may be used to control the operation of the optical elements 112, and thereby control with precision the delivery of the light from the light source 110 to the cornea 2 by controlling any combination of: wavelength, bandwidth, intensity, power, location, depth of penetration, and duration of treatment. In addition, the function of the controller 120 can be partially or wholly replaced by a manual operation. For example, the applicator 132 can be manually operated to deliver the cross-linking agent 130 to the cornea 2 without the assistance of the controller 120. In addition, the controller 120 can operate the applicator 132 and the optical elements 112 according to inputs dynamically supplied by an operator of the delivery system 100 in real time, or can operate according to a pre-programmed sequence or routine.

Other aspects of devices and approaches for applying a cross-linking agent to the cornea and delivering light to activate the applied cross-linking agent are described in U.S. patent application Ser. No. 13/051,699, filed Mar. 18, 2011, the contents of which are incorporated entirely herein by reference.

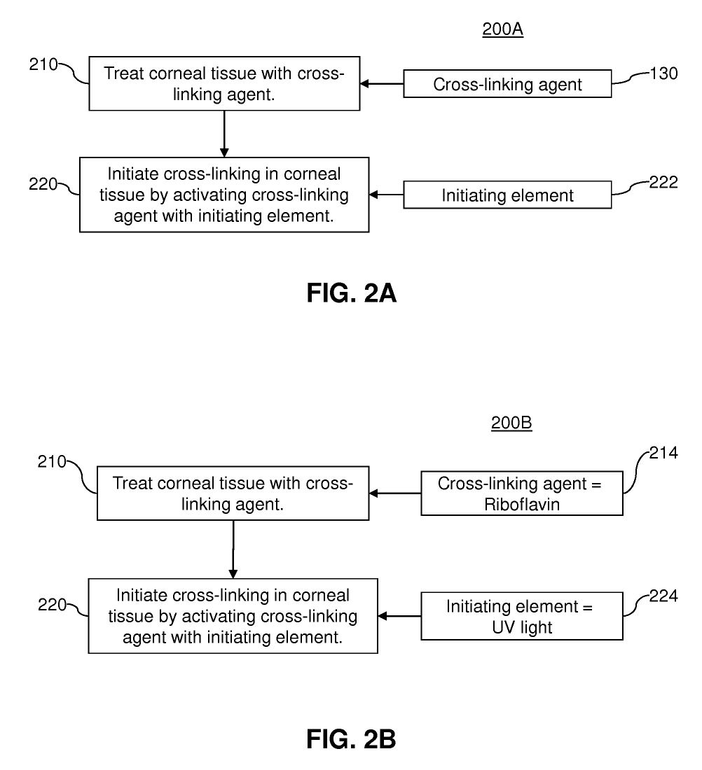

FIGS. 2A-2B, which describe an exemplary operation of the delivery system 100, where the cross-linking agent 130 is applied to the cornea 2 using the applicator 132. Once the cross-linking agent 130 has been applied to the cornea 2, the cross-linking agent 130 is initiated by light from the light source 110 to cause cross-linking agent 130 to absorb enough energy to release free oxygen radicals within the cornea 2. Once released, the free oxygen radicals (i.e. singlet oxygen) form covalent bonds between corneal collagen fibrils and thereby cause the corneal collagen fibrils to cross-link and change the structure of the cornea 2. For example, activation of the cross-linking agent 130 with the light source 110 delivered to the cornea 2 through the optical elements 112 may result in cross-linking in the mid-depth region 2B of the cornea 2 and thereby strengthen and stiffen the structure of the cornea 2.

Referring to FIG. 2A, an example embodiment 200A according to aspects of the present disclosure is illustrated. Specifically, in step 210, the corneal tissue is treated with the cross-linking agent 130. Step 210 may occur, for example, after a treatment is applied to generate structural changes in the cornea and produce a desired shape change. Alternatively, step 210 may occur, for example, after it has been determined that the corneal tissue requires stabilization or strengthening. The cross-linking agent 130 is then activated in step 220 with an initiating element 222. Activation of the cross-linking agent 130, for example, may be triggered thermally by the application of microwaves or light. In an example configuration, the initiating element 222 may be the light from the light source 110 shown in FIG. 1.

As the example embodiment 200B of FIG. 2B shows further, Riboflavin may be applied topically as a cross-linking agent 214 to the corneal tissue in step 210. As also shown in FIG. 2B, ultraviolet (UV) light may be applied as an initiating element 224 in step 220 to initiate cross-linking in the corneal areas treated with Riboflavin. Specifically, the UV light initiates cross-linking activity by causing the applied Riboflavin to release reactive oxygen radicals in the corneal tissue. In particular, the Riboflavin acts as a sensitizer to convert O₂ into singlet oxygen which causes cross-linking within the corneal tissue.

The cross-linking agent 130 may be applied to the corneal tissue in an ophthalmic solution, e.g., from an eye dropper. In some cases, the cross-linking agent 130 is effectively applied to the corneal tissue after removal of the overlying epithelium. However, in other cases, the cross-linking agent 130 is effectively applied in a solution that transitions across the epithelium into the underlying corneal tissue, i.e., without removal of the epithelium. For example, a transepithelial solution may combine Riboflavin with approximately 0.1% benzalkonium chloride (BAC) in distilled water. Alternatively, the transepithelial solution may include other salt mixtures, such as a solution containing approximately 0.4% sodium chloride (NaCl) and approximately 0.02% BAC. Additionally, the transepithelial solution may contain methyl cellulose, dextran, or the like to provide a desired viscosity that allows the solution to remain on the eye for a determined soak time.

The amount of time required to achieve the desired cross-linking can be controlled by adjusting the parameters for delivery and activation of the cross-linking agent. In an example implementation, the time can be reduced from minutes to seconds. While some configurations may apply the initiating element (e.g., from the light source 110) at a flux dose of 5 J/cm², aspects of the present disclosure allow larger doses of the initiating element, e.g., multiples of 5 J/cm², to be applied to reduce the time required to achieve the desired cross-linking. Highly accelerated cross-linking is particularly possible with the devices and approaches described in detail in U.S. application Ser. No. 13/051,699, filed Mar. 18, 2011, referenced above.

To decrease the treatment time, and advantageously generate stronger cross-linking within the cornea 2, the initiating element (e.g., the light source 110) may be applied with a power between 30 mW and 1 W. The total dose of energy absorbed in the cornea 2 can be described as an effective dose, which is an amount of energy absorbed through a region of the corneal surface 2A. For example the effective dose for a region of the cornea 2 can be, for example, 5 J/cm², or as high as 20 J/cm² or 30 J/cm². The effective dose delivering the energy flux just described can be delivered from a single application of energy, or from repeated applications of energy. In an example implementation where repeated applications of energy are employed to deliver an effective dose to a region of the cornea 2, each subsequent application of energy can be identical, or can be different according to information provided by a feedback system.

Although LASIK surgery may initially achieve desired reshaping of the cornea 2, the desired effects of reshaping the cornea 2 may be mitigated or reversed at least partially if the collagen fibrils within the cornea 2 continue to change after the desired reshaping has been achieved. Indeed, complications may result from further changes to the cornea 2 after treatment. For example, a complication known as post-LASIK ectasia may occur due to the permanent thinning and weakening of the cornea 2 caused by LASIK surgery. In post-LASIK ectasia, the cornea 2 experiences progressive steepening (bulging).

Therefore, aspects of the present disclosure preserve the desired corneal structure and shape that result from LASIK surgery. In particular, embodiments provide approaches for initiating molecular cross-linking of the corneal collagen to stabilize the corneal tissue and improve its biomechanical strength and stiffness after the desired shape change has been achieved. In addition, embodiments may provide devices and approaches for monitoring cross-linking in the corneal collagen and the resulting changes in biomechanical strength to provide a feedback to a system for inducing cross-linking in corneal tissue. Such devices and approaches are described in detail in U.S. application Ser. No. 13/051,699, filed Mar. 18, 2011, referenced above.

As described previously, during LASIK surgery, an instrument called a microkeratome is used to cut through the corneal epithelium to form a thin flap. The flap is then peeled back and the underlying cornea tissue is reshaped using an excimer laser so that light traveling through the cornea is properly focused onto the retina. In one embodiment, the outer surface of the cornea, e.g., in the area of the flap, is treated with a cross-linking agent, e.g., Riboflavin, after the flap is put back in place. The cross-linking agent is then activated with an initiating element. Activation of the cross-linking agent, for example, may be triggered thermally by the application of microwaves or light to corresponding areas of the cornea. Cross-linking occurs in the area of application. Although the cross-linking agent is applied to the outer surface of the cornea, i.e., the epithelium, it has been shown that cross-linking agents can chemically transition across the outer surface into the underlying corneal tissue, i.e., the stroma. Thus, in some embodiments, the cross-linking agent may be delivered to the underlying corneal tissue by applying the cross-linking agent topically to the outer surface of the cornea. Moreover, in further embodiments, the outer surface may be treated to promote the transition of the cross-linking agent therethrough.

In another embodiment, after the flap is peeled back, inner surfaces of the cornea are exposed for the application of a cross-linking agent. In particular, the inner surface of the flap as well as the underlying corneal tissue are exposed. Therefore, the inner surface of the flap and/or the underlying corneal tissue are treated with a cross-linking agent. In other words, the cross-linking agent may be applied to (i) the inner surface of the flap only, (ii) the underlying corneal tissue only, or (iii) both the inner surface of the flap and the underlying corneal tissue. The cross-linking agent is then activated with an initiating element. Again, activation of the cross-linking agent may be triggered thermally by the application of microwaves or light. Although the initiating element may be applied before the flap is put back, the initiating element additionally or alternatively may be applied to the treated areas after the flap is put back in place. In this case, the initiating element can be delivered through the outer surface of the cornea.

According to yet another embodiment, the inner surface of the flap and/or the underlying corneal tissue are treated with a cross-linking agent after the flap is peeled back. The cross-linking agent is then activated with an initiating element. As in the previous embodiment, the cross-linking agent may be applied to (i) the inner surface of the flap only, (ii) the underlying corneal tissue only, or (iii) both the inner surface of the flap and the underlying corneal tissue. In addition, the outer surface of the cornea, e.g., in the area of the flap, is treated with a cross-linking agent after the flap is put back in place. The cross-linking agent is then activated in step with an initiating element. Again, activation of the cross-linking agent may be triggered thermally by the application of microwaves or light. In a variation of this embodiment, the cross-linking agent may be activated with an initiating element according to a single act, rather than two separate acts. Thus, the initiating element may be delivered in the single act after the flap is put back in place.

Accordingly, a cross-linking agent may be applied and activated in different regions at different points during LASIK treatment. For example, the cross-linking agent may be applied to any combination of the outer surface of the cornea, the inner surface of the flap, and the exposed underlying corneal tissue. Moreover, specially tailored concentrations of cross-linking agent may be applied in combination with varying levels of initiating element to these regions to achieve the appropriate amount of stability and strength in the cornea.

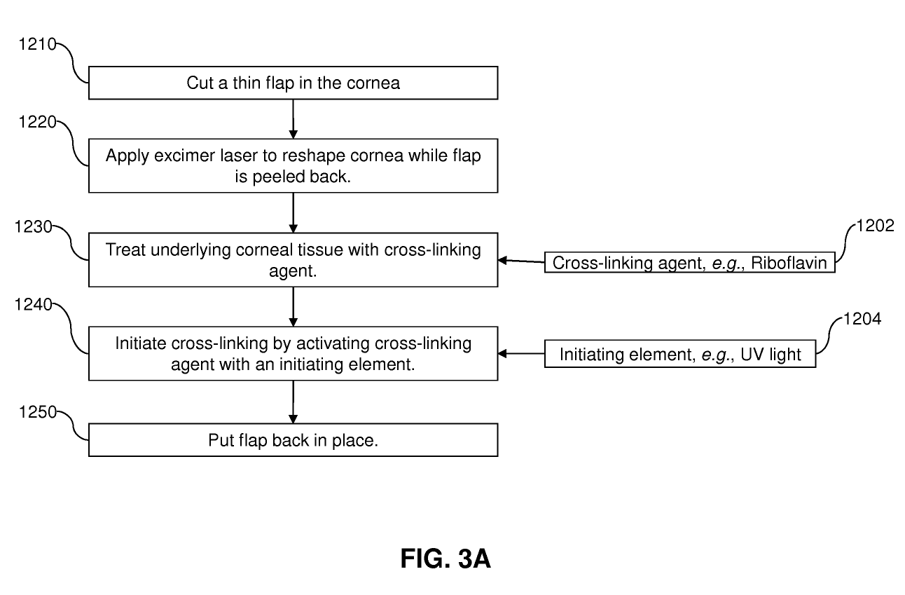

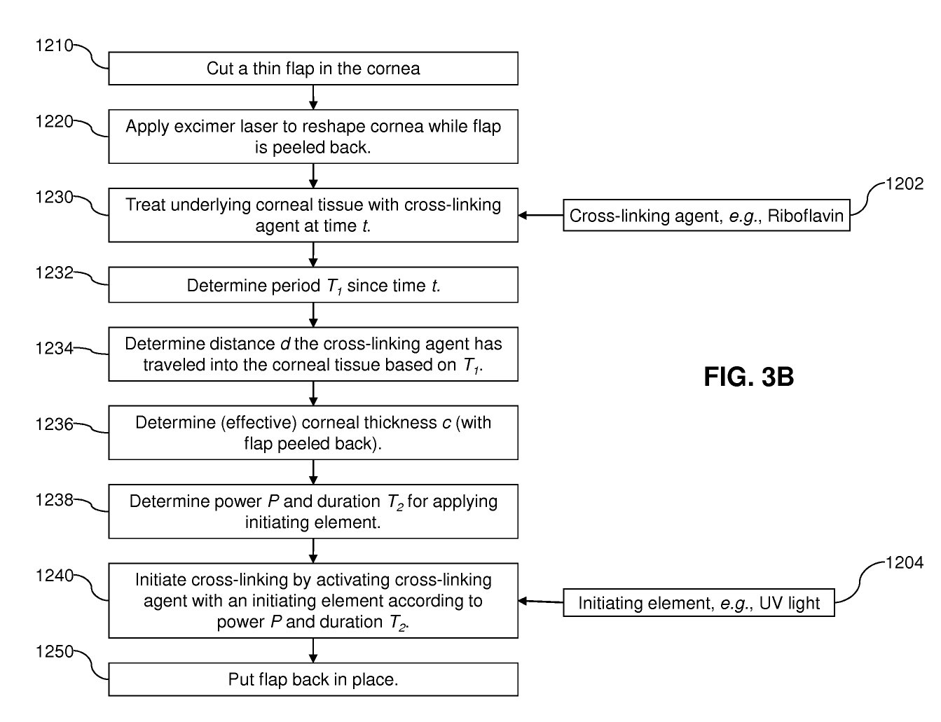

FIG. 3A illustrates the activation of cross-linking in the regions of the cornea treated by the LASIK surgery. After the flap is peeled back in act 1210, inner surfaces of the cornea are exposed for the application of a cross-linking agent. In particular, the underlying corneal tissue is exposed. Therefore, in act 1230, the underlying corneal tissue is treated with a cross-linking agent 1202 after the excimer laser reshapes the cornea in act 1220. The cross-linking agent 1230 may be applied, for example, by dripping a measured amount and concentration of the cross-linking agent 1202 topically onto the exposed underlying corneal tissue. The cross-linking agent 1202 is then activated in act 1240 with an initiating element 1204 while the flap remains peeled back. Activation of the cross-linking agent 1202 may be triggered thermally by the application of microwaves or light. In step, 1250, the flap is put back in place.

In an example embodiment, Riboflavin may be applied as the cross-linking agent 1202 to the corneal tissue. In addition, a photoactivating light, such as ultraviolet (UV) light, may be applied as an initiating element 1204 to initiate cross-linking in the corneal areas treated with Riboflavin. To achieve optimal results, an appropriate amount of Riboflavin is applied to the targeted regions of the cornea and an appropriate amount of UV light is applied to match the application of Riboflavin. In some cases, damage to the eye may result if too much Riboflavin and UV light reach the endothelium. This may occur, in particular, if too much time passes between the application of the Riboflavin in act 1230 and the application of the UV light in act 1240. The passage of time allows the Riboflavin to diffuse more deeply into the corneal tissue to the endothelium, and the UV light may reach the Riboflavin at the endothelium.

Thus, according to aspects of the present invention further illustrated in FIG. 3B, embodiments apply the UV light in act 1240 according to a power and duration that ensure that the application of the UV light does not damage the endothelium. The resulting energy determines the depth to which the UV light penetrates in the corneal tissue. The power and duration are based in part on the amount of time that has passed since the application of the cross-linking agent. Because the rate of diffusion for a given concentration of the cross-linking agent is known, embodiments can use the time data to calculate how far the cross-linking agent has traveled into the corneal tissue. Therefore, in act 1232, a time period T₁ is determined from the time t when the cross-linking agent 1202 is applied in act 1230. Based on this time period T₁, a distance d representing how far the cross-linking agent has traveled into the corneal tissue is determined in act 1234.

Moreover, the power and duration are also based on the distance that the cross-linking agent and the UV light can travel though the cornea before reaching the endothelium. This distance generally corresponds with the thickness of the cornea. Therefore, in act 1236, the corneal thickness c is determined. By determining the diffusion distance d of the cross-linking agent and determining the thickness c of the cornea, the appropriate power P and duration T₂ can be determined in act 1238. The embodiments can apply the UV light to the cross-linking agent in the cornea in act 1240 while preventing damage to the endothelium.

When the cross-linking agent is applied and activated while the corneal flap remains peeled back during LASIK surgery, the amount of corneal tissue through which the cross-linking agent and the UV light can travel decreases. The risk of damage to the endothelium may be greater during LASIK surgery. Thus, to ensure that the UV light does not reach the endothelium, act 1236 may determine an “effective” thickness c by adjusting for the fact that the corneal flap is peeled back. For example, peeling back the flap may reduce the effective thickness of the cornea to 120 μm, and embodiments may apply the UV light according to a power P and duration T₂ that delivers the UV light to a depth of 100 μm.

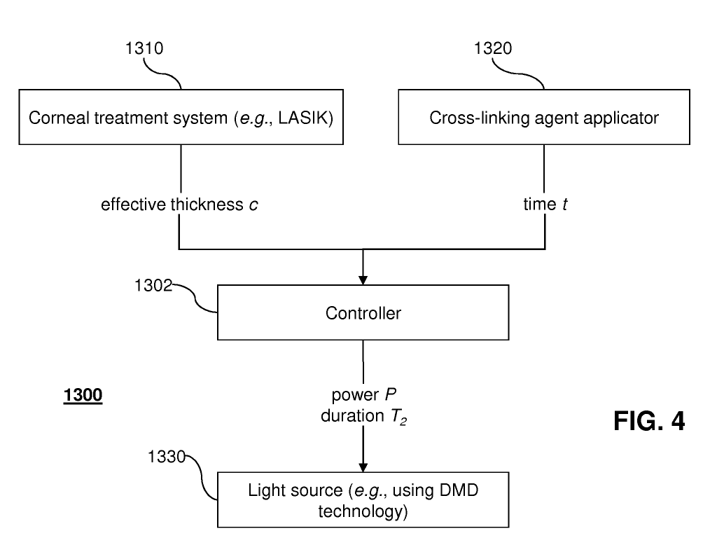

Referring to FIG. 4, an embodiment 1300 may employ a corneal treatment system 1310 (e.g., for applying LASIK surgery), a cross-linking agent applicator 1320 (e.g., for applying Riboflavin to the cornea), and a light source 1330 (e.g., for delivering the UV light to the cornea). Advantageously, aspects of the present invention integrate the operation of the treatment system 1310, the cross-linking agent applicator 1320, and the light source 1330. A controller 1302, e.g., a computer or other processing device, receives input data from the treatment system 1310 and the cross-linking agent applicator 1320 and determines parameters for the operation of the light source 1330. In particular, the controller 1302 determines the appropriate power P and duration T₂ for the controlled light source 1330 to apply light to activate the cross-linking agent.

Referring to FIG. 4, the treatment system 1310 creates a flap that is peeled back and reduces the cornea's effective thickness c, and the cross-linking agent applicator applies the cross-linking agent at a particular time t. The controller 1302 receives the effective thickness c and the time t as input data. Applying the process described in FIG. 3B, the controller 1302 then determines the appropriate power P and duration T₂ for the application of the light by the light source 1330.

While embodiments above apply Riboflavin in combination with LASIK surgery to stabilize and strengthen the corneal tissue, aspects of these embodiments may also be employed to apply Riboflavin to reduce the risk of infection when the outer protective layer of the eye is compromised during LASIK surgery. The Riboflavin acts as a photosensitizer that increases the absorption of UV light. The resulting absorption of UV light can induce DNA and RNA lesions, and as a result, is effective in killing viruses, bacteria, and other pathogens in the field.

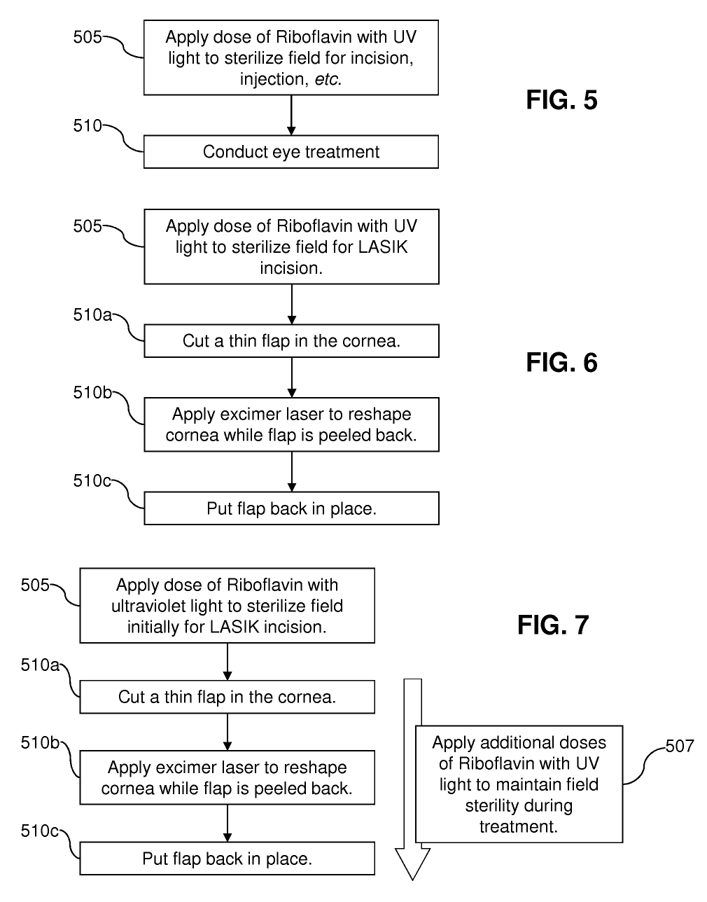

Referring to FIG. 5, an example embodiment prophylactically applies in vivo a combination of Riboflavin and ultraviolet (UV) light in act 505 to sterilize the field prior to the eye treatment in act 510, which involves incision, injection, or other penetration of the outer protective layer of the eye. The amount of Riboflavin and the exposure to the UV light is sufficient to achieve sterility in the field while minimizing any damage or other unwanted effects in the tissue. In some cases, the dosage of UV light may be equal to or exceed the dosage typically applied when inducing cross-linking activity to strengthen and stabilize corneal tissue in a treatment. In some cases, for example, higher powers of UV light and shorter exposure times may be preferred.

LASIK surgery is a particular invasive treatment that compromises the outer protective layer of the eye. Referring to the example embodiments of FIGS. 6-8, LASIK surgery is employed to produce a desired reshaping of the cornea. Specifically, as shown in FIG. 6, a microkeratome is used to cut through the corneal epithelium and form a thin flap in the cornea in act 510a. In act 510b, the flap is peeled back and the underlying corneal tissue is reshaped by the application of an excimer laser. After the desired reshaping of the cornea is achieved, the cornea flap is put back in place in act 510c to complete the surgery. To reduce the risk of infection when the microkeratome cuts through the epithelium, Riboflavin and ultraviolet (UV) light are applied prophylactically in act 505 to sterilize the field prior to the treatment.

Referring to FIG. 7, the embodiment applies one or more additional amounts of Riboflavin with UV light in act(s) 507 at selected times to maintain sterility of the field during the treatment, i.e., acts 510a-c. The appropriate amounts of Riboflavin solution may be applied in spurts during the treatment.

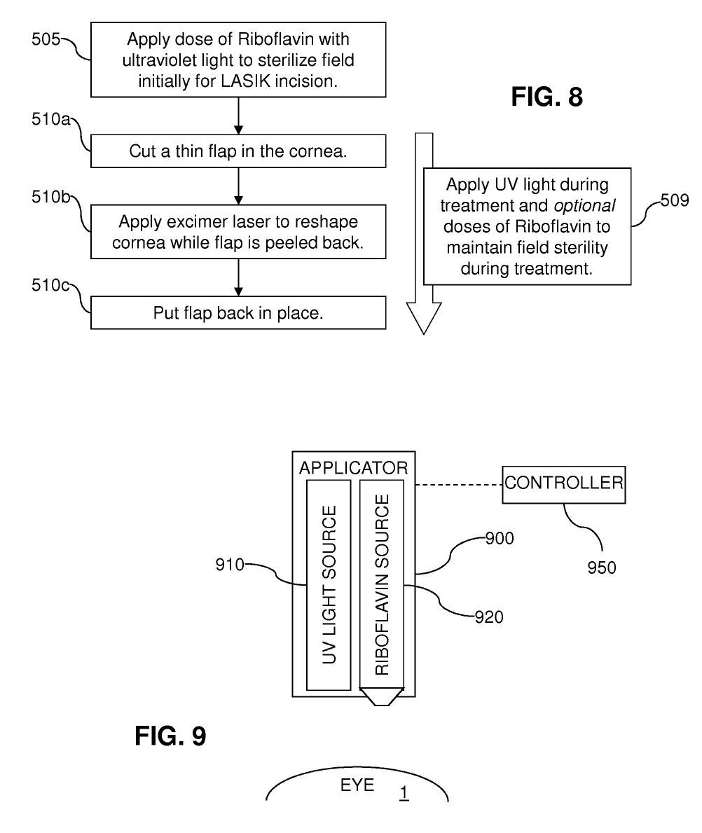

Referring to FIG. 8, the embodiment applies UV light in act 509 throughout the treatment, i.e., acts 510a-c. Riboflavin has already been applied with UV light in act 505, and as such, it may be sufficient to apply just UV light throughout the treatment to maintain sterility. Riboflavin, however, may be optionally applied, e.g., via spurts, during the treatment if necessary.

Referring to FIG. 9, an integrated applicator 900 for delivering the Riboflavin with UV light to an eye 1 is illustrated. In particular, the applicator 900 combines a first component 910 for applying Riboflavin and a second component 920 for applying UV light. The first component 910 and the second component 920 are configured to direct the Riboflavin and the UV light to the desired area on the eye 1. In one example, the first component 910 may be a pressurized nozzle that delivers measured amounts of Riboflavin and the second component 920 may be an light emitting diode (LED) or a like source for UV light. According to one implementation, the applicator 900 is positioned over the field of the eye and is operated to apply UV illumination and spurts of Riboflavin during the treatment, as described with reference to FIGS. 7 and 8. Aspects of the applicator 900 may be manually operated by the practitioner, and/or may be automated and controlled, for example, with a controller 950. Systems and approaches described in detail in U.S. application Ser. No. 13/051,699, filed Mar. 18, 2011, may be employed with the controller 950 to monitor and control the sterilizing application of the Riboflavin and delivery of the UV light during the LASIK surgery.

The present disclosure includes systems having controllers for providing various functionality to process information and determine results based on inputs. Generally, the controllers (such as the controller 120 or 950) may be implemented as a combination of hardware and software elements. The hardware aspects may include combinations of operatively coupled hardware components including microprocessors, logical circuitry, communication/networking ports, digital filters, memory, or logical circuitry. The controller may be adapted to perform operations specified by a computer-executable code, which may be stored on a computer readable medium.

As described above, the controller 120 or 950 may be a programmable processing device, such as an external conventional computer or an on-board field programmable gate array (FPGA) or digital signal processor (DSP), that executes software, or stored instructions. In general, physical processors and/or machines employed by embodiments of the present disclosure for any processing or evaluation may include one or more networked or non-networked general purpose computer systems, microprocessors, field programmable gate arrays (FPGA's), digital signal processors (DSP's), micro-controllers, and the like, programmed according to the teachings of the exemplary embodiments of the present disclosure, as is appreciated by those skilled in the computer and software arts. The physical processors and/or machines may be externally networked with the image capture device(s) used for monitoring, etc., or may be integrated to reside within the image capture device. Appropriate software can be readily prepared by programmers of ordinary skill based on the teachings of the exemplary embodiments, as is appreciated by those skilled in the software art. In addition, the devices and subsystems of the exemplary embodiments can be implemented by the preparation of application-specific integrated circuits or by interconnecting an appropriate network of conventional component circuits, as is appreciated by those skilled in the electrical art(s). Thus, the exemplary embodiments are not limited to any specific combination of hardware circuitry and/or software.

Stored on any one or on a combination of computer readable media, the exemplary embodiments of the present disclosure may include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, and the like. Such computer readable media further can include the computer program product of an embodiment of the present disclosure for performing all or a portion (if processing is distributed) of the processing performed in implementations. Computer code devices of the exemplary embodiments of the present disclosure can include any suitable interpretable or executable code mechanism, including but not limited to scripts, interpretable programs, dynamic link libraries (DLLs), Java classes and applets, complete executable programs, and the like. Moreover, parts of the processing of the exemplary embodiments of the present disclosure can be distributed for better performance, reliability, cost, and the like.

Common forms of computer-readable media may include, for example, a floppy disk, a flexible disk, hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read.

While the present disclosure has been described in connection with a number of exemplary embodiments, and implementations, the present disclosure is not so limited, but rather cover various modifications, and equivalent arrangements.

What is claimed is:

1. A method for treating an eye, comprising:
applying, to an outer protective layer of an area of an eye, one or more initial doses of a cross-linking agent that acts as a photosensitizer;
delivering, from a light source, one or more initial doses of ultraviolet light to the area of the eye, the cross-linking agent increasing absorption of the ultraviolet light by the area of the eye, the absorption of the ultraviolet light sterilizing the area of the eye before the outer protective layer is penetrated;
penetrating the outer protective layer of the eye to provide access to an area below the outer protective layer; and
applying a treatment to the eye via the access provided by cutting the outer protective layer.

2. The method of claim 1, further comprising delivering, from the light source, one or more additional doses of the ultraviolet light after penetrating the outer protective layer of the eye, the one or more additional doses of the ultraviolet light further sterilizing the area of the eye.

3. The method of claim 2, further comprising applying, one or more additional doses of the cross-linking agent after penetrating the outer protective layer of the eye, the one or more additional doses of the cross-linking agent increasing absorption of the ultraviolet light by the area of the eye, the absorption of the ultraviolet light further sterilizing the area of the eye.

4. The method of claim 3, further comprising monitoring the cross-linking agent applied to the area of the eye and determining when to apply the one or more additional doses of the cross-linking agent and the one or more additional doses of the ultraviolet light.

5. The method of claim 1, wherein penetrating the outer protective layer of the eye includes cutting a thin flap from the outer protective layer of a cornea, and applying the treatment to the eye includes reshaping the cornea with an excimer laser while the thin flap is peeled back.

6. The method of claim 1, wherein the cross-linking agent is Riboflavin.

7. The method of claim 1, wherein the one or more initial doses of the cross-linking agent are applied in spurts from a pressurized nozzle.

8. The method of claim 3, wherein the one or more additional doses of the cross-linking agent are applied in spurts from a pressurized nozzle.

9. The method of claim 1, wherein the light source is an light emitting diode.

10. The method of claim 1, wherein the one or more initial doses of the ultraviolet light provides a total effective dose of greater than approximately 5 J/cm².

11. The method of claim 1, wherein a controller determines the application of the one or more initial doses of the cross-linking agent and the one or more initial doses of the ultraviolet light.

12. The method of claim 3, wherein a controller determines the application of the one or more initial doses of the cross-linking agent, the one or more additional doses of the cross-linking agent, the one or more initial doses of the ultraviolet light, and the one or more additional doses of the ultraviolet light.
